(19) United States
(12) Patent Application Publication
(10) Pub. No.: US 2007/0270916 A1
(43) Pub. Date: Nov. 22, 2007

(54) Cardiac pacemaker with integrated battery

(76) Inventors: Robert E. Fischell, Dayton, MD (US); Kristina M. Johnson, Durham, NC (US)

Correspondence Address: Dr. Robert E. Fischell, 14600 Viburnum Drive, Dayton, MD, 21036, US

(21) Appl. No.: 11/436,176

(22) Filed: May 18, 2006

(51) Int. Cl. A61N 1/00

(52) U.S. Cl. 607/36

(57) ABSTRACT

Disclosed is any implanted medical device (an IMD) that has either a primary or a rechargeable battery that has a case that forms part of the case of the implant. Furthermore, the case of the battery is typically welded to a second metal can that would generally enclose the electronic components of the implant. This construction provides the thinnest case so as to be surgically placed under the skin of the chest with the least bulging. Also disclosed is the concept of coating the IMD and any electrical lead that comes out of the IMD with an antibiotic and/or anti-inflammatory coating so as to minimize any post-implant infection or inflammation.

### FIELD OF USE

This invention is in the field of devices for implantation in a human patient to treat a medical condition of that patient.

### BACKGROUND OF THE INVENTION

There are many medical devices that are implanted in patients to treat a variety of human disorders. Examples of such devices are cardiac pacemakers, implantable cardioverter defibrillators, vagus nerve stimulators, electrical stimulators for the brain and spinal chord, coronary ischemia detectors, etc. Each of these devices is powered by a battery that is placed inside the outer case of that implant. None of these devices utilize the case of the battery as also being the case of the implant itself. Having two cases, one inside the other, increases the thickness of the implant which is undesirable.

In U.S. Pat. No. 3,867,950, Robert E. Fischell describes a rechargeable cardiac pacemaker that utilizes a rechargeable nickel-cadmium cell. This cell chemistry has the disadvantage of a high rate of self-discharge at body temperature. It is desirable to recharge a rechargeable pacemaker as infrequently as possible. Therefore, using rechargeable cells that are now available in the 21^{st} century, such as the lithium-ion cells, and others that are currently known in the art offer a great advantage over earlier rechargeable cells that have a high rate of self-discharge.

To be safe and effective for human use, a rechargeable pacemaker should indicate to the patient when recharging is necessary. That attribute has never been used with any prior rechargeable implant. Furthermore, any implanted device is improved if it includes a means for informing the patient, or his or her doctor or a medical condition monitoring facility about vital functions of such an implant. No rechargeable pacemaker has been described that can inform the patient in whom the device is implanted that the battery needs recharging and can also warn the patient if there is any other pacer related attribute about which he/she should be alerted.

### SUMMARY OF THE INVENTION

The present invention is any implanted medical device (an IMD) that has either a primary or a rechargeable battery that has a case that forms part of the case of the implant. Furthermore, the case of the battery is typically welded to a second metal can that would generally enclose the electronic components of the implant. Besides welding, other means for joining the battery case to the case that surrounds the electronics section, such as silver soldering or soldering or an adhesive bond, could be used. The design concept of the case of the battery forming part of the case of the IMD provides the smallest possible thickness which allows the device to be less protruding under the patient's skin. This is particularly valuable for certain people who are very thin. That is because it is easy to hide even a comparatively thick implant in a person who has a considerable amount of fat, but to avoid excess bulging in a thin person, it is highly desirable to have the IMD be as thin as possible.

For the purposes of this specification, a rechargeable pacemaker will be described in detail. However, it should be understood that any IMD can use the inventive concepts that are taught in this specification and the invention can be used with either primary or rechargeable batteries. Examples of such other devices that can utilize this concept are implantable cardioverter defibrillators, vagus nerve stimulators, brain stimulators, implanted ischemia detectors, spinal chord stimulators, etc. In point of fact, all the inventive concepts described herein could be used with any type of IMD.

Another important feature of the present invention is the ability to alert the patient or the patient's caretaker or the patient's doctor or a centrally located diagnostic center of a variety of conditions that relate to the operation of the IMD. This warning or alerting can be accomplished by a vibration generator such as is used with cell phones, by an audio signal or by electrical stimulation which is frequently termed an “electrical tickle.” As described in U.S. Pat. No. 6,985,771 by R. E. Fischell et al, the patient alert for recharging or any other function of any IMD that requires attention could also be sent to an external device that is easily accessible by the patient or it could be sent to some monitoring location that is remote from the patient. A unique feature described herein is to use different signal patterns for an implanted pacemaker to inform the patient that 1) the rechargeable cell needs recharging; 2) the heart is not captured by the electrical pulse of the pacemaker; 3) there is an increase in the humidity within the pacemaker that is indicative of a potential failure of the device; and 4) any other attribute of the IMD for which the patient should be notified. It is envisioned that each of these conditions could be indicated by a different alerting pattern.

Another important feature of the device is that the pacemaker's outer surface and the lead that connects the pacemaker to the patient's heart could be treated with an antibiotic material that either elutes over time or is permanently attached to the surfaces of the pacemaker can and the lead. A very effective antibiotic coating would be one that combines an eluting antibiotic drug that elutes for a time period of a few days to a few months and a second coating that is antibiotic that remains permanently fixed to the pacemaker's surface. Such coatings that elute or remain permanently fixed to the surface of the implant have been used for many years with devices such as stents and central venous catheters. However, such coatings have never been used with an implanted device such as a pacemaker. Another attribute of the coating can be that it is anti-inflammatory which would serve to decrease inflammation in the region where the pacemaker and lead are implanted.

Still another inventive feature of the pacemaker is having the proximal portion of the lead permanently attached to the plastic header of the pacemaker. The prior art pacemakers use a lead that has to be attached at the header by means of at least one set screw. Since all pacemakers used today have primary batteries that are depleted in a time period of a few years or as many as 12 years, it has been necessary for the lead to be allowed to remain in place while the pacemaker itself is replaced. However, if a rechargeable pacemaker is used that will last the patient's lifetime, there is no need for ever separating the lead from the pacemaker. Therefore, one aspect of the present invention envisions that the lead may be permanently attached to the plastic header of the pacemaker. With such a permanent attachment, the header can be made considerably smaller and thinner as compared to the size of a header that would include means for attaching and removing the lead.

Still another feature of the pacemaker described herein is to have the lead joined to the pacemaker through a strain relief section that emerges from the header of the pacemaker case parallel to the case's outer surface. This is different from most pacemakers where the lead emerges from the pacemaker not parallel to the outer perimeter of the pacemaker. Since the excess pacemaker lead has to be wrapped around the pacemaker's outer case, having the strain relief of the lead emerge from the header parallel to the pacemaker outer surface is advantageous for easily wrapping excess length of lead around the pacemaker's perimeter.

To be most adaptable for the needs of different patients, the pacemaker should be programmable from an external programmer. To prevent inadvertent changes in the pacemaker's operating parameters, the circuitry inside the pacemaker that allows for reprogramming of the pacemaker parameters would ideally be turned on only when there is charging current being placed into the pacemaker's rechargeable battery. For the pacemaker of the present invention, the pacemaker parameters that would be programmable include the following: (1) stimulation pulse voltage; (2) stimulation pulse wave duration; (3) voltage level for the electrical tickle to alert the patient; (4) the voltage of the battery that triggers the alerting system to alert the patient; (5) enable or disable the alarm for humidity detection; (6) turn on time period for enabling long range telemetry It is anticipated that additional parameters of the pacemaker may be programmable in order to optimize the pacemaker's performance capability.

The pacemaker of the present invention would typically have telemetry turned on when the pacemaker is being charged. One design feature of the pacemaker is that long range telemetry could remain on without recharging for a set turn on time period. That period of time could be set by an internal timer or the telemetry could be turned off by the external programmer. Parameters that would typically be measured by telemetry include: (1) battery voltage; (2) battery charge current during recharging; (3) battery discharge current during normal operation; (4) pulse voltage setting; (5) pulse time duration setting; (6) whether or not the heart is captured by the stimulation pulse; (6) signal from the heart indicating if the lead is broken; (7) level of humidity detected inside the electronics can; (8) any comparatively high electrical resistance that indicates the start of a short circuit; (9) the level of the voltage for the electrical tickle alerting of the patient; and (10) any other parameter that is useful to telemeter out of the implant.

Thus one object of the present invention is to have an IMD that has either a primary or a rechargeable battery whose outer case permanently forms part of the case of the IMD.

Another object of this invention is that the IMD is a pacemaker.

Another object is that the IMD is an implantable cardioverter defibrillator.

Another object is that the IMD is an implantable detector of coronary ischemia.

Still another object of this invention is to have a rechargeable pacemaker that has a rechargeable battery whose case forms part of the case of the pacemaker.

Still another object of this invention is that the IMD is an implantable cardioverter defibrillator.

Still another object of this invention is to have a rechargeable pacemaker that has an alerting indicator for the patient that can alert the patient or caretaker or a remote monitoring site if the battery needs recharging or if there is any other aspect of the pacemaker's functioning for which the patient should be alerted.

Still another object of the invention is to have alerting electrodes on the IMD's outer surface to provide an electrical tickle to inform the patient of any aspect of the IMD's performance that should be noted.

Still another object of the invention is to have the voltage level of the electrical tickle adjustable so that it is clearly discernable by the patient but not painful.

Still another object of this invention is to have a different alerting signal for each different condition of the IMD that causes an alerting signal to be created.

Still another object of this invention is having an IMD whose electrical lead emerges from the device's header with a strain relief that runs generally parallel to the IMD's outer case.

Still another object of this invention is to utilize a metal for the IMD's can that is an alloy having a high electrical resistivity so as to decrease case heating during the recharging process.

Still another object of this invention is to have a lead whose proximal end is permanently attached through a strain relief to a plastic header that is part of the IMD.

Still another object of the invention is to disallow reprogramming of the IMD except when the battery is being recharged by magnetic induction through the skin.

Still another object of the invention is to provide telemetry of various parameters of the IMD.

Still another object of the invention is to have an IMD whose operating parameters are programmable from an external programmer only during the time that the battery is being recharged.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 is a plan view of a rechargeable IMD that has its recharging coil placed around the periphery of the case of the IMD.

FIG. 2 is a cross section of the MD at section 2-2 of FIG. 1.

FIG. 3 is a top view of the rechargeable IMD showing the location of electrodes for providing electrical tickle alerting to alert the patient regarding how the patient's IMD is operating.

FIG. 4 illustrates a patient being recharged by an externally located recharger.

FIG. 5 illustrates a patient being recharged by an externally located, portable recharger.

FIG. 6 is an electrical block diagram for the rechargeable IMD system.

### DETAILED DESCRIPTION OF THE INVENTION

Throughout this detailed description of the invention, the invented device will usually be described as a cardiac pacemaker. However, it should be understood that most of the inventive concepts described for the pacemaker could also be used for a variety of IMDs such as those previously described herein. Furthermore, it should be understood that many of the features of the IMD described herein could be achieved with either a rechargeable or a primary battery.

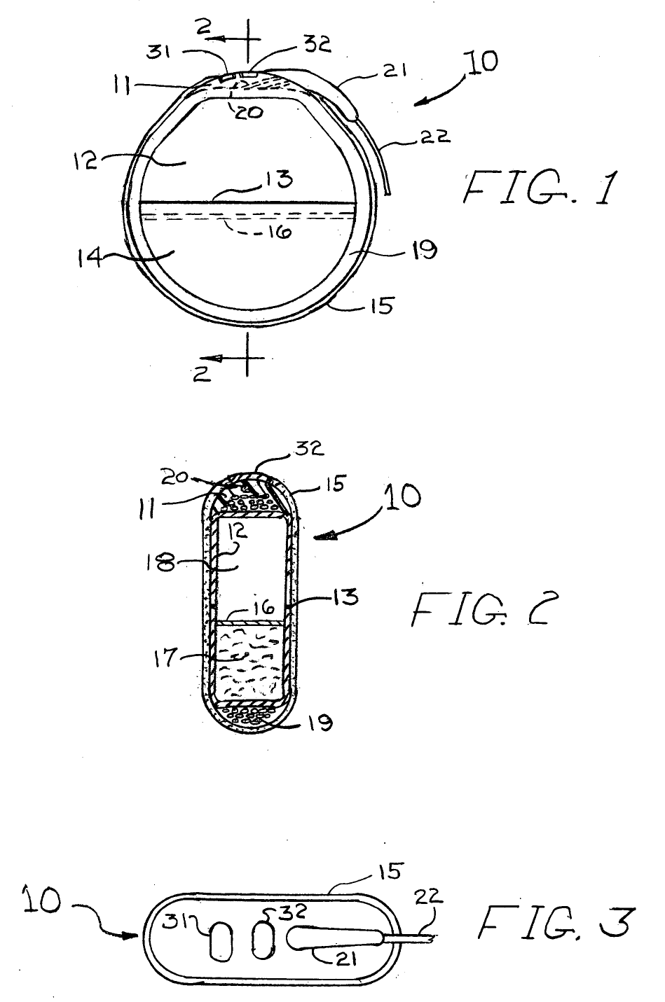

FIGS. 1, 2 and 3 illustrate a rechargeable pacemaker 10 having a plastic header 11, an electronics can 12, an electronics-battery case weld 13, a battery can 14, a pacer anti-biotic coating 15, a battery cover seal 16, a rechargeable battery 17, an electronics section 18, a recharging pick-up coil 19, lead wires 20 within the header 11, a strain relief 21 for the electrical lead 22 and electrical tickle alerting electrodes 31 and 32.

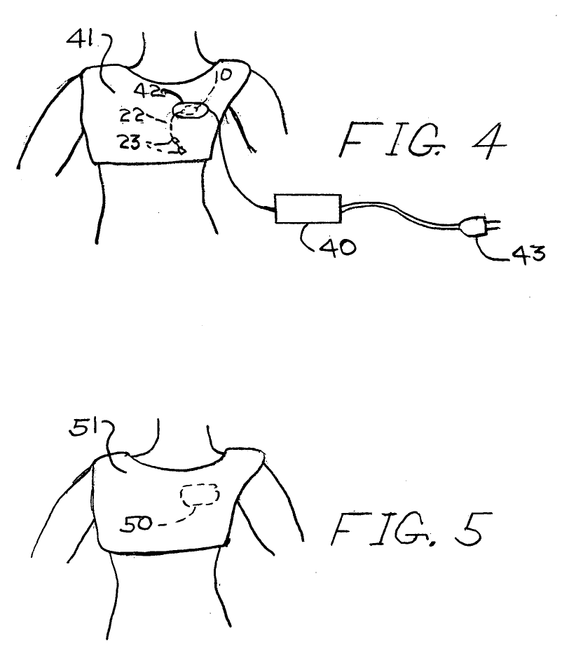

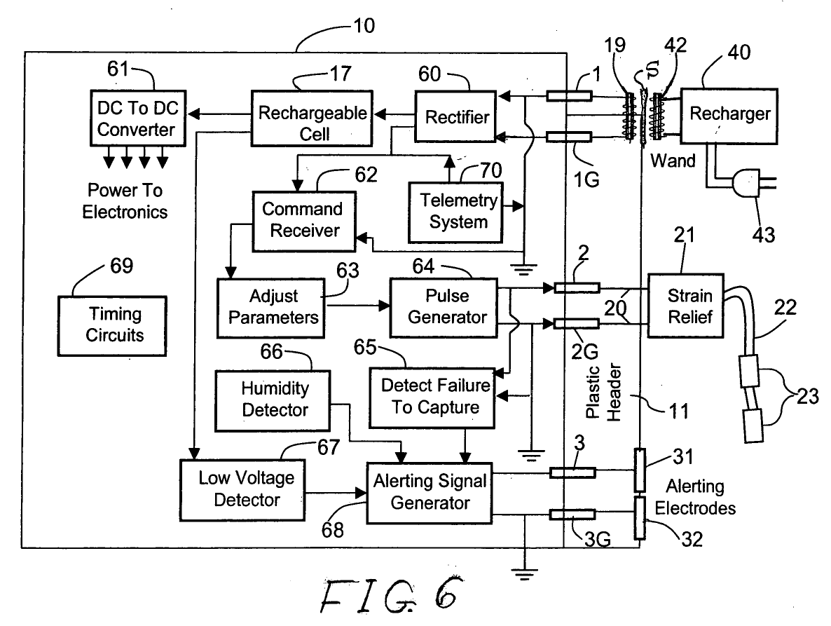

The header 11 is molded from a firm plastic such as polycarbonate and contains the interface between the lead wires 20 and the electronics can 12. As can be seen in FIG. 2, the pick-up coil 19 is also contained within the header 11. The pick-up coil 19 should be molded in the same plastic material as the header 11. By not being within the metal case of the pacemaker 10, the coil will not experience a Faraday shield effect so it will take a lower level alternating magnetic field impressed on the coil 19 by the charger 40 of FIG. 4 or the charger 50 of FIG. 5 to properly charge the rechargeable battery within the pacemaker 10. It would be typical to have hermetically sealed feed-thrus (not shown) from the header into electronics can 12 to electrically connect the pacemaker electronics shown in FIG. 6 to the lead wires 20 which join through the strain relief 21 to the lead 22 that goes to the patient's heart.

The electronics can 12 would be typically joined by a weld 13 to the battery can 14. If not by welding, any other attachment means that sealably and fixedly attaches the cans 12 and 14 together is envisioned. The attachment must be a hermetic seal to keep out body fluids and to securely join the two cans together. As can be seen in FIG. 2, the can 12 contains the electronic section 18 of the pacemaker 10. It is envisioned that the electronics could be encapsulated in a plastic that is coated with a metal or glass that creates a hermetic seal with the case of the battery. For the purposes of this specification of the invention, all such configurations that would provide a hermetic seal for the electronics would be regarded as a “metal can.”

A novel feature of the design of this pacemaker is the recharging pick-up coil 19 that (as can be seen in FIGS. 1 and 2) is encapsulated in plastic and placed around the perimeter of battery can 14 and the electronics can 12. Prior pick-up coils for receiving energy by magnetic induction from an external source of electric power were typically placed inside of a rechargeable pacemaker. The novel coil 19 of the present invention can receive electrical energy with less heating of the metal case of the pacemaker. To limit heating of the metal cans 12 and 14, the metal should have a comparatively high resistivity. An ideal metal for the cans 12 and 14 would be the alloy titanium 6-4. Other alloys which could be used are those of stainless steel which have a high electrical resistivity.

As can be seen in FIG. 1, the strain relief 21 emerges from the plastic header 11 so as to be generally parallel to the outer perimeter of the pacemaker 10. This allows any excess lead wire 22 to be easily wrapped around the perimeter of the pacemaker 10.

The greatest risk to a patient after having any IMD implanted is infection in the pocket where the IMD is placed and infection along the lead into the heart. In the USA, the typical procedure to avoid infection is to give a bolus injection of an antibiotic such as cephalosporin both before and at eight hours after the implant procedure. Additionally, before closing, the implanting physician would irrigate the pocket with an antibiotic such as neomycin. Still further, the patient would take an oral antibiotic such as Keflex for several weeks post-procedure.

To further reduce the risk or prevent and control post-operative inflammation and infection, the pacemaker casing and catheter can be coated with an antibiotic, anti-microbial, anti-inflammatory or other agents to reduce the risk of infection and/or inflammation in and around the pocket created for the pacemaker and the region surrounding the catheter. These agents can either remain on the surface of the pacemaker or catheter or they could be slowly released by elution over a relatively short period of time following implant. Such surface treatment and/or elution of the appropriate drug(s) could reduce the probability of occurrence for the most common complication of the implant surgery which is infection in the pocket. The entire case or at least a portion of the case could be coated with these antibiotic agents. The lead and strain relief can also be coated to reduce or prevent catheter-related bloodstream infections. These infections should be avoided because they are expensive to treat due to pharmacy charges, lead changes, lab tests, and extended hospital stays. By eluting over a period of about one month post-procedure, the eluting antibiotic agent would be acting locally and effectively long after the bolus injections and irrigation have provided their protection against infection. Depending on the drug that is used, elution over a time period that is shorter or longer than approximately one month can be accomplished by varying the polymer into which the drug is placed or varying the amount any/or type of polymer that is used to coat the IMD.

FIGS. 1 and 2 show an outer surface antibiotic coating 15 which can contain the antibiotic agent that either elutes slowly through a polymer that contains that agent or it is permanently attached to that outer surface. Optimally, one antibiotic agent would elute through the coating 15 and another antibiotic agent would be permanently fixed to the outer surface of the coating 15.

Coatings 15 to be used for the pacemaker case and lead or to elute from the surfaces include, but are not limited to, the following agents: the antibiotics cephalosporin, neomycin, Keflex, minocycline and rifampin and the antiseptic agents chlorhexidrine and silver sulfadiazine to guard against infection. Any one of these agents can be placed into or onto a polymer coating of the pacemaker that could be either bioabsorbable or permanently remain on the pacemaker's surface.

In any case, since infection in the pocket of the pacemaker 10 is the most prevalent failure mode for implanted IMDs, having an antibiotic layer on the outer surface of the IMD and the strain relief 21 and the lead 22 should significantly reduce the in-pocket and lead infection rate. This would be particularly valuable where pacemakers are implanted under less than optimal sterility settings.

To avoid post-operative scarring of the skin at the implant site, an ointment containing an anti-scarring agent could be used. Drugs such as, but not limited to, sirolimus have proved efficacious as an anti-inflammatory and anti-proliferative agent for drug-eluting stents. Any drug of the sirolimus family could be used in the form of an ointment to be placed over the incision for the pacemaker to reduce post-operative scarring at that location.

FIGS. 1 and 3 show alerting electrodes 31 and 32 that can provide the patient with an electrical tickle, to warn the patient of a variety of conditions relative to the operation of the implanted pacemaker 10. The pacemaker 10 could be programmed by the patient's doctor or a technician to set the voltage level for electrical tickle that was clearly discernible by the patient but not painful. One type of alerting that could be provided by electrical tickle would inform the patient when the rechargeable battery needed to be recharged. Another warning could be programmed to alert the patient if the energy of the electrical pulse from the pacemaker 10 was insufficient to capture the heart; i.e., a higher voltage and/or longer pulse duration would be needed to cause the heart to beat. In the event of lead breakage, the electrical tickle would alert the patient that there is no pulse energy being delivered into the heart. In that case, the pacemaker 10 and lead 22 would have to be explanted. Still another warning could be the presence of increased humidity within the electronics section 18 which could be the result of a defective feed-through. Such a warning would allow the pacemaker 10 to be replaced before the occurrence of an undesirable failure of the pacemaker's electronics. Any important change in the pacemaker's electrical circuitry that would be predictive of a failure to pace could also be sensed and the appropriate electrical tickle could be provided.

FIG. 4 illustrates a patient having the implanted pacemaker 10 recharged by means of an external recharger 40 that has a charging wand 42 and has a plug 43 for plugging into a source of electrical power. The wand 42 could have a Velcro attachment to the vest 41 at a place that is directly over the site of the implanted pacemaker 10. Alternatively, the wand 42 could be placed in a pocket in the vest 41. When there is sufficient electrical power going into the rechargeable battery 17, the recharger 40 would include a visual display or an audio message indicating that the recharging current into the battery was correct. The recharger 40 could also indicate by audio and/or visual means when the recharging of the rechargeable battery was completed. These features would be accomplished by telemetry as explained with the assistance of FIG. 6. FIG. 4 also shows the lead 22 having bipolar electrodes 23 that are used to stimulate the heart to make it beat appropriately. Although the design for pacing a single chamber of the heart is shown, it should be understood that multi-chamber pacing could be accomplished with the implanted pacemaker 10 as described herein. Also monopolar as well as bipolar leads could be used.

Recharging the battery of the IMD by magnetic induction is a most practical method to accomplish replenishment of the battery's power. However, alternative methods for recharging including having electrodes that are on the skin to which the battery and a source of recharging power could be attached is certainly envisioned. Electrodes made from pyrolitic carbon are well known to be accepted by the skin in a manner similar to finger nails. Therefore, such a method, as well as other methods for recharging are envisioned.

FIG. 5 illustrates a vest 51 that has a pocket or Velcro connection means that holds a portable recharger 50. This portable recharger 50 would have all the operating characteristics of the charger 40 except that it would derive its operating power from a battery that could be rechargeable or replaceable. The portable charger 50 would allow the patient to be recharged without any significant restraint on the patient's activities. For example, use of the portable recharger 50 would allow the patient to recharge while performing activities such as housework, gardening, working at a job, etc. Thus, longer recharge times (such as 2 to 5 hours) could be accomplished without any inconvenience for the patient because the patient's mobility would not be compromised.

FIG. 6 is a block diagram of the electronic circuitry for the pacemaker 10. The external recharger 40 which is powered through the plug 43 provides an alternating electric current into the coil of the wand 42 that produces an alternating magnetic field. This alternating magnetic field at a frequency between 2 and 500 kHz is transmitted through the skin S and is picked up by the pick-up coil 19 which recharges the rechargeable battery 17 through the rectifier 60. If desired, the output of the rectifier 60 could be filtered to smooth the voltage fed into the battery 17. Since the coil 19 is external to the case of the pacemaker 10, hermetic seal feed-thrus 1 and 1G are used to bring the power into the electronic section 18 of the pacemaker 10. Although the frequency for the magnetic induction charging of the battery 17 could be between 1 and 500 kHz, and optimum frequency is in the range of 10 to 50 kHz. Although only the recharger 40 is shown in FIG. 6, it should be understood that the portable recharger 50 could also be used for powering the pacemaker 10 while allowing greater mobility for the patient during the recharging process. The rechargeable battery 17 feeds into a DC to DC converter 61 that provides a variety of voltages to operate the pacemaker 10.

FIG. 6 also shows that the command receiver 62 and telemetry system 70 are powered from the output of the rectifier 60. Having the command receiver 62 powered only during charging disallows inadvertent reprogramming of the pacemaker parameters. Not shown in FIG. 6 is a digital memory within the command receiver 62 that retains the operating parameters that have been programmed into the pacemaker 10 through the command receiver 62. Although the telemetry system 70 would be turned on when magnetic induction power is turned on, a timer (not shown) within the telemetry system 70 could keep telemetry on for a set period of time after the charging magnetic field was removed. This would allow for a period of time when long-range telemetry could be used when the patient was not connected to a recharger. The output signal from the telemetry system 70 would typically be through the electrodes 1 and 1G and radiate from the coil 19 that would act as an antenna.

The command receiver 62 would have an input into the adjust parameters circuitry 63 that is capable of adjusting all the parameters of the pacemaker 10. The pulse generator 64 would have its parameters, such as pulse voltage and pulse duration, adjusted by an input from the adjust parameters circuitry 63. The output of the pulse generator 64 would be through the feed-thrus 2 and 2G to the wires 20 within the plastic header 11. The wires 20 connect through the strain relief 21 and the lead 22 to the electrodes 23. The detect failure to capture circuitry 65 would be capable of detecting if the pulse energy is sufficient to cause the heart to beat. If the lead 22 was broken, the failure to capture alarm would occur. The alerting signal generator 68 would send an alerting signal through the feed-thrus 3 and 3G to the alerting electrodes 31 and 32 if the circuitry 65 detected that the pulse from the pulse generator 64 was not causing the heart to beat. The humidity detector 66 and the battery low voltage detector 67 would also send an alerting electrical tickle signal through the electrodes 31 and 32 if either the humidity was too high inside the electronics can 12 or if the battery voltage was too low. The battery voltage too low alert could serve as a reminder to the patient to recharge the pacemaker's battery 17.

It should be noted that the electrodes 1G, 2G and 3G are all connected to ground and therefore they could in fact be a single electrode. Furthermore, the ground could be the case of the pacemaker 10 so that the grounded electrode 32 could in fact be some bare portion of the outer surface of the pacemaker 10.

The timing circuit 69 shown in FIG. 6 would provide all the timing necessary to operate the circuitry of all the pacemaker electronics.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

What is claimed is:

1. An implantable medical device that includes a battery that has a metal can whose external surface constitutes a portion of the external surface of the device, the battery's metal can being sealably and fixedly attached to another metal can that generally surrounds the electronic section of the implantable medical device.

2. The implantable medical device of claim 1 where the battery is a rechargeable battery that is recharged from a recharger that is located external to the body of the human patient.

3. The implantable medical device of claim 2 where the recharger is battery operated to allow mobility for the patient during the recharging process.

4. The implantable medical device of claim 2 where the rechargeable battery is a lithium ion cell.

5. The implantable medical device of claim 2 where the frequency for recharging lies between 1.0 kHz and 500 kHz.

6. The implantable medical device of claim 1 where the metal can for the battery is formed from an alloy of either titanium or iron.

7. The implantable medical device of claim 1 where the implantable medical device is selected from the group consisting of pacemakers, implantable cardioverter defibrillators, brain stimulators, spinal nerve stimulators, vagal nerve stimulators and devices that can detect coronary ischemia.

8. The implantable medical device of claim 1 including a plastic header into which the strain relief for the electrical leads into the heart is permanently attached.

9. The implantable medical device of claim 8 where the strain relief emerges from the implantable medical device so as to be generally parallel to the outer surface of the implantable medical device.

10. The implantable medical device of claim 1 including means for alerting the patient as to various operating conditions of the implantable medical device.

11. The implantable medical device of claim 10 where the means for alerting the patient is selected from the group consisting of an audio signal, vibration of the implantable medical device and an electrical tickle.

12. The implantable medical device of claim 10 where there are at least two different alerting signals that correspond to at least two different operating conditions for which the patient should be alerted.

13. The implantable medical device of claim 10 including means for alerting the patient in whom the device is implanted if a rechargeable battery within the implantable medical device requires recharging.

14. The implantable medical device of claim 10 including means for alerting the patient in whom the device is implanted if one of the operating conditions for which the patient should be alerted is an actual or potential failure of the device to function properly.

15. The implantable medical device of claim 14 also including alerting when the battery requires recharging and the alerting signal is different for at least two different indications for which the patient should be alerted.

16. The implantable medical device of claim 1 including a polymer coating on the device's outer surface that includes an antibiotic agent.

17. The implantable medical device of claim 16 where the antibiotic agent elutes through the polymer coating over a time period between a few days to several months.

18. The implantable medical device of claim 1 including a command receiver for programming the parameters of the device, the command receiver being turned on only when there is recharging current being applied to a rechargeable battery within the device.

19. A pacemaker for pacing the heart of a human patient, the pacemaker including a rechargeable cell that is recharged by magnetic induction though the patient's skin from a recharger that is located outside of the body of the human patient, the rechargeable cell having a biocompatible metal case which constitutes a portion of the outer surface of the pacemaker, the batteries metal case being sealably and fixedly attached to a metal case that generally encloses the electronic section of the pacemaker.

20. A method for decreasing the formation of scar tissue that forms at the incision over the pocket into which an IMD is placed, the method including placing an ointment containing sirolimus or an analog of sirolimus onto the incision after it is closed and then placing a sterile bandage over the ointment at the site of the incision.
